(19) United States
(12) Patent Application Publication
(10) Pub. No.: US 2007/0225629 A1
(43) Pub. Date: Sep. 27, 2007

(54) ORTHOPEDIC BRACE

(75) Inventors: James ISRAEL, Somerset, NJ (US); Juan PAEZ, Rockaway, NJ (US)

Correspondence Address: HARNESS, DICKEY & PIERCE, P.L.C., P.O. BOX 828, BLOOMFIELD HILLS, MI, 48303, US

(73) Assignee: EBI, L.P., Parsippany, NJ (US)

(21) Appl. No.: 11/685,221

(22) Filed: Mar. 13, 2007

Related U.S. Application Data
(60) Provisional application No. 60/784,506, filed on Mar. 21, 2006.
(60) Provisional application No. 60/796,606, filed on May 1, 2006.
(60) Provisional application No. 60/889,438, filed on Feb. 12, 2007.

(51) Int. Cl. A61F 5/00

(52) U.S. Cl. 602/21; 602/20; 602/12; 602/5

(57) ABSTRACT

A brace for a patient appendage. The brace can include a first member and a second member. The first member is positioned proximate a first side of the patient appendage and includes a main body portion extending in a longitudinal direction. The first member additionally includes a circumferentially extending portion at least partially extending about the patient appendage. The second member is positioned proximate a second side of the patient appendage and includes a first end having an outer convexly curved portion.

### CROSS-REFERENCE TO RELATED APPLICATIONS

This application claims the benefit of U.S. Provisional Application No. 60/784,506, filed Mar. 21, 2006. This application claims the benefit of U.S. Provisional Application No. 60/796,606, filed May 1, 2006. This application claims the benefit of U.S. Provisional Application No. 60/889,438, filed Feb. 12, 2007. The disclosures of the above applications are incorporated herein by reference.

### INTRODUCTION

Various orthopedic or orthotic braces are known in the art for facilitating healing of injured or strained portions of arms, legs, hands, fingers or other appendages.

The present teachings provide a brace that is convenient to use and can be adapted to the anatomy of a patient's appendage.

### SUMMARY

According to one aspect, the present teachings are directed to a brace for a patient appendage, the brace includes a first member and a second member. The first member is positioned proximate a first side of the patient appendage and includes a main body portion extending in a longitudinal direction. The first member additionally includes a circumferentially extending portion at least partially extending about the patient appendage. The second member is positioned proximate a second side of the patient appendage and includes a first end having an outer convexly curved portion.

According to another aspect, the present teachings are directed to a brace for an arm of a patient. The brace includes a palmar member and a dorsal member. The palmar member is positioned proximate a palmar side of the patient arm. The palmar member includes a main body portion extending in a longitudinal direction and a circumferentially extending portion for at least partially extending about the patient arm. The dorsal member is positioned proximate a dorsal side of the patient appendage. The dorsal member includes a first end having an outer convexly curved portion.

According to yet another aspect, the present teachings provide a method of bracing a patient appendage. The method includes providing a first member having a first end and a second end. The first end includes a circumferentially extending portion. The first member is positioned on the appendage such that the circumferentially extending portion at least partially extends circumferentially about the appendage. The method additionally includes providing a second member. The second member includes a first end having an outer convex surface.

According to another aspect, the present teachings are directed to a brace for an appendage of a patient. The brace includes a first member and a second member. The first member may be positioned proximate a first side of the patient appendage and may extend in a longitudinal direction. The second member may be positioned proximate a second side of the patient appendage. At least one of the first member and the second member may include at least one trim line extending generally in the longitudinal direction and providing a guide for adjusting a circumferential size of the brace.

Further areas of applicability of the present teachings will become apparent from the description provided herein. It should be understood that the description and specific examples are intended for purposes of illustration only and are not intended to limit the scope of the present teachings.

### DRAWINGS

The present teachings will become more fully understood from the detailed description and the accompanying drawings, wherein:

FIG. 1 is a side view of a brace constructed in accordance with the teachings of the present disclosure, the brace shown operatively associated with an appendage;

FIG. 2 is an exploded perspective view of the brace of FIG. 1;

FIG. 3 is a side view of a first member of the brace of FIG. 1;

FIG. 4 is a perspective view of the first member of the brace of FIG. 1;

FIG. 5 is a bottom view of a second member of the brace of FIG. 1.

FIG. 6 is a side view of another brace constructed in accordance with the teachings of the present disclosure, the brace shown operatively associated with an appendage;

FIG. 7 is an exploded view of the brace of FIG. 6;

FIG. 8 is a top perspective view of a first member of the brace of FIG. 6;

FIG. 9 is a bottom view of the first member of the brace of FIG. 6; and

FIG. 10 is a cross-sectional view taken along the line 10-10 of FIG. 6.

### DESCRIPTION OF VARIOUS ASPECTS

The following description of the present teachings will be understood to be merely exemplary in nature and is in no way intended to limit the invention, its application, or uses.

The brace 10 may be used to promote healing of a fracture, or support for strain or pain relief, for example. In one particular application, the brace 10 may be used to promote healing of a pediatric, distal radius fracture such as a non-displaced torus fracture or other pediatric or adult applications.

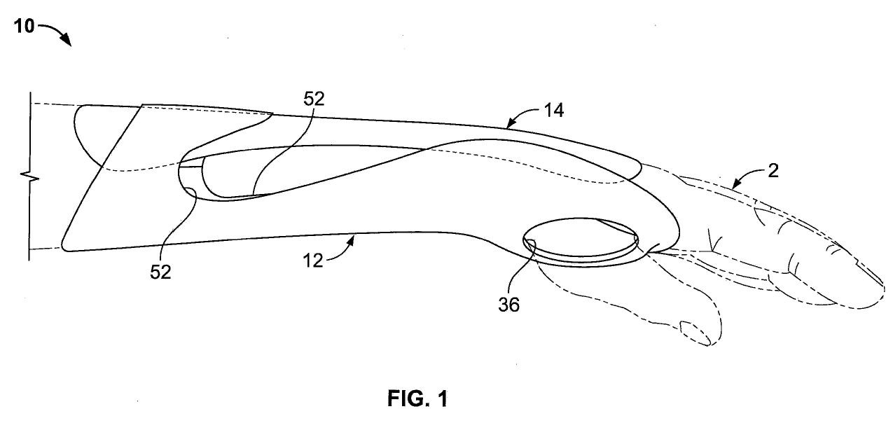

With general reference to FIGS. 1-5 of the drawings, a brace in accordance with the present teachings is illustrated and generally identified at reference character 10. As shown, the brace 10 may be particularly adapted for use on a hand, wrist and forearm to promote healing. In other applications, the teachings may be adapted for other appendages of the body. As such, it will be understood that the present teachings have applications beyond the exemplary embodiments illustrated in the drawings.

The brace 10 may generally include a first member or palmar member 12 and second member or dorsal member 14. The first and second members 12 and 14 may be positioned on opposite sides of an appendage 2. As shown in FIG. 1, the first member 12 may be positioned adjacent a palmar side of the hand and corresponding portions of the wrist and forearm.

The first member 12 may include a main body portion 16. The main body portion 16 may extend longitudinally between a first end or proximal end 18 and a second end or distal end 20. The main body portion 16 may include a concavely curved inner surface 22 for receiving the patient appendage 2.

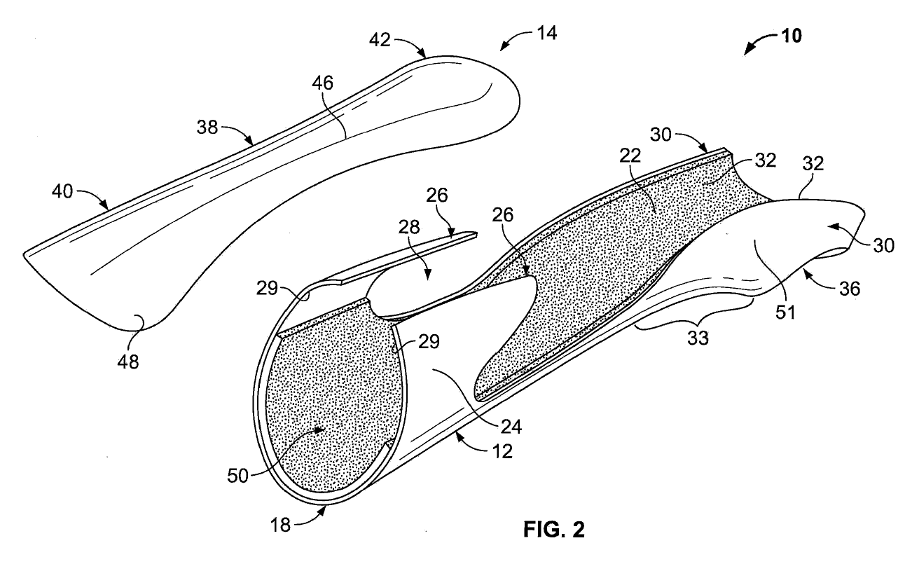

The first member 12 may additionally include a circumferentially extending portion 24 for at least partially extending circumferentially 24 about the appendage 2. The circumferentially extending portion 24 may be positioned proximate the first end of the brace 10 and may extend more than 180 degrees (i.e., more than half way) about the appendage 2. As illustrated, the circumferentially extending portion 24 may not completely extend about the appendage 2. In this manner, the circumferentially extending portion 24 includes a pair of ends or arms 26 spaced apart by a gap 28 (see FIG. 2, for example). The circumferentially extending portion 24 may extend through approximately 340 degrees about the appendage 2, although it can also extend 360 degrees about the appendage 2. The arms 26 of the circumferentially extending portion 24 may define concavely curved inner surface 29.

As illustrated, the first member 12 may include a pair of circumferential extensions 30 proximate the second end 20. The extensions 30 may wrap more than half way around the appendage 2. The extensions 30 may define concavely curved inner surfaces 32.

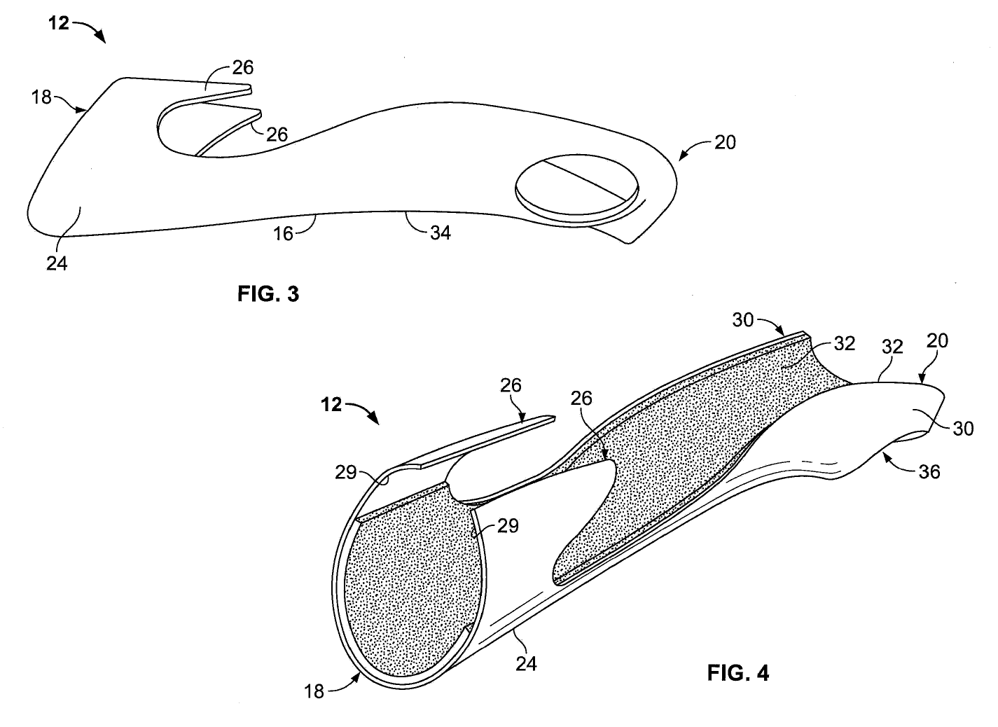

The second end 20 of the first member 12 may include a first flared area 33 (see FIG. 2). The first flared area 33 may be positioned proximate a base of the thumb. The second end 20 may additionally include a second flared area 34 (see FIG. 3) proximate the ulnar styloid. The second end 20 may additionally define a recess 36 for receiving the thumb. The recess may be an aperture 36.

The second member 14 may be positioned adjacent a dorsal side of the hand and corresponding portions of the wrist and forearm. The second member 14 may include a central portion 38 extending between a first end or proximal end 40 and a second end or distal end 42. The central portion may be a narrowing section 38. The first and second ends 40 and 42 may widen as they extend away from the narrowing section 38.

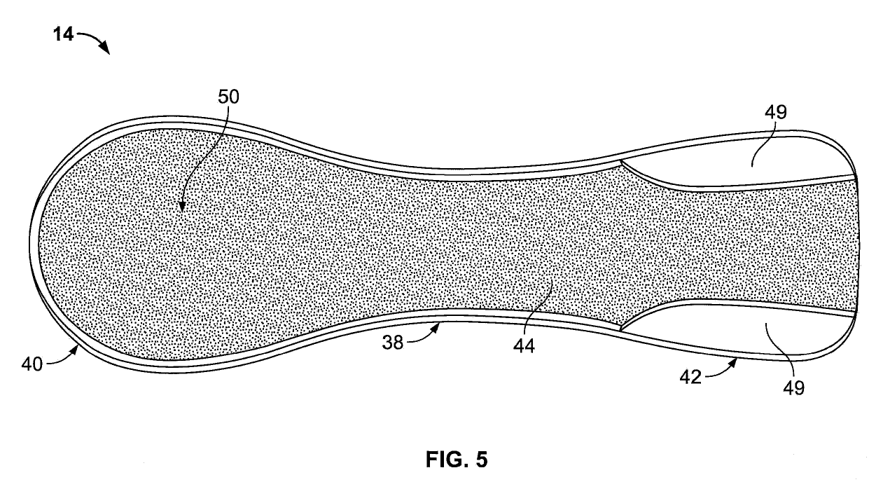

The second member 14 may include a first side 44 (see FIG. 5) for positioning adjacent the appendage 2 and an opposing second side 46 (see FIG. 2). The first side 44 may be concavely curved about the longitudinal axis. Additionally, the dorsal member may be curved in a direction from proximal to distal as depicted in FIG. 1. The second side 46 may be convexly curved about the longitudinal axis. The second side 46 of the first end 40 may define surfaces 48 (see FIG. 2) for mating with concavely curved inner surfaces 29 of the circumferentially extending portion 24. The first side 44 of the second end 42 may define surfaces 49 (see FIG. 5) for mating with the convexly curved outer surfaces 51 of the extensions 30.

The first and second members 12 and 14 may be constructed of plastic, such as polyethylene, for example, or other suitable material. The inner surfaces of the first and second members 12 and 14 may be provided with a breathable cushioning material 50. This cushioning material 50 may be removed at the mating surfaces of the first and second members 12 and 14. The cushioning material 50 may be adhesively or otherwise secured to the first and second members 12 and 14.

In use, the first member 12 may be positioned about the appendage 2. The thumb may extend through the aperture 36. In certain applications, the circumferentially extending portion 24 may be positioned proximate to a fracture of the appendage 2.

The second member 14 is placed adjacent the appendage 2 but distally to the orientation shown in FIG. 1. The second member 14 may be translated proximally such that the first end 40 of the second member 14 is received by the mating area of the first member 12. The widest portion of the second member 14 may be positioned proximal to the arms 26 defined by the circumferentially extending portion 24. The mating area of the second member 14 at its second end 42 may be positioned above the extensions 30 of the first member 12. The second member 14 may resist distal movement/migration. In this regard, distal movement/migration of the second member 14 would push the arms 26 apart.

Upon assembly, the first and second members 12 and 14 cooperate to define laterally oriented openings 52 (see FIG. 1). The openings 52 improve ventilation to the appendage 2. The openings 52 may be bigger or smaller than these shown in the drawings. Additional openings can also be included.

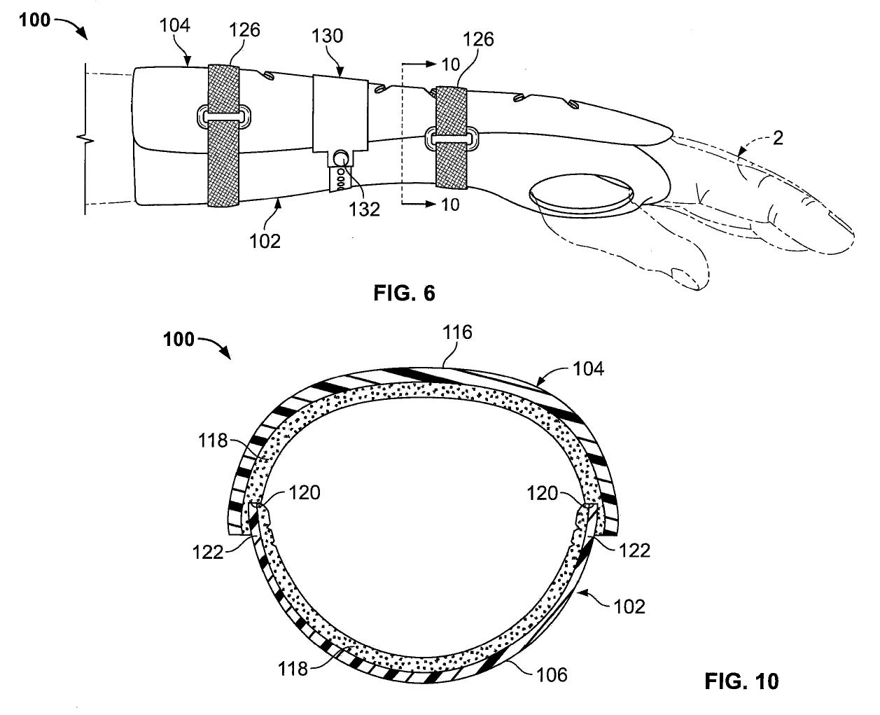

In certain applications, a strap may be used near the second end 20 of the first member 12 to further secure the brace 10 to the appendage 2, such as strap 126 illustrated in FIG. 6 in connection with another brace 100. In other applications, the second member 14 may include an extension (not shown) that wraps at least partially around the aperture 36 or the appendage 2 to interlock with the first member 12 and fix the second member 14 relative to the first member 12.

With the relative positions of the first and second members 12 and 14 fixed, the brace 10 may behave as a single part. The brace 10 may have a certain stiffness generated through its geometry (i.e., its assembled geometric moment of inertia) that is greater than a stiffness generated by the geometry of traditional clamshell type braces.

As illustrated throughout FIGS. 1-5, portions of the brace 10 may wrap more than 180 degrees about the appendage 2 and cooperate with mating portions of the brace 10. In this regard, the arms 26 and the extensions 30 of the first member 12 may wrap more than 180 degrees about the appendage 2 and cooperate with the second member 14. Alternatively or additionally, the first end 40 of the second member 14, the second end 42 of the second member 14, or both ends 40 and 42 may wrap more than 180 degrees about the appendage 2 and cooperate with the first member 12. It will be appreciated by those skilled in that art that the cooperating portions of the first and second members 12 and 14 are interchangeable.

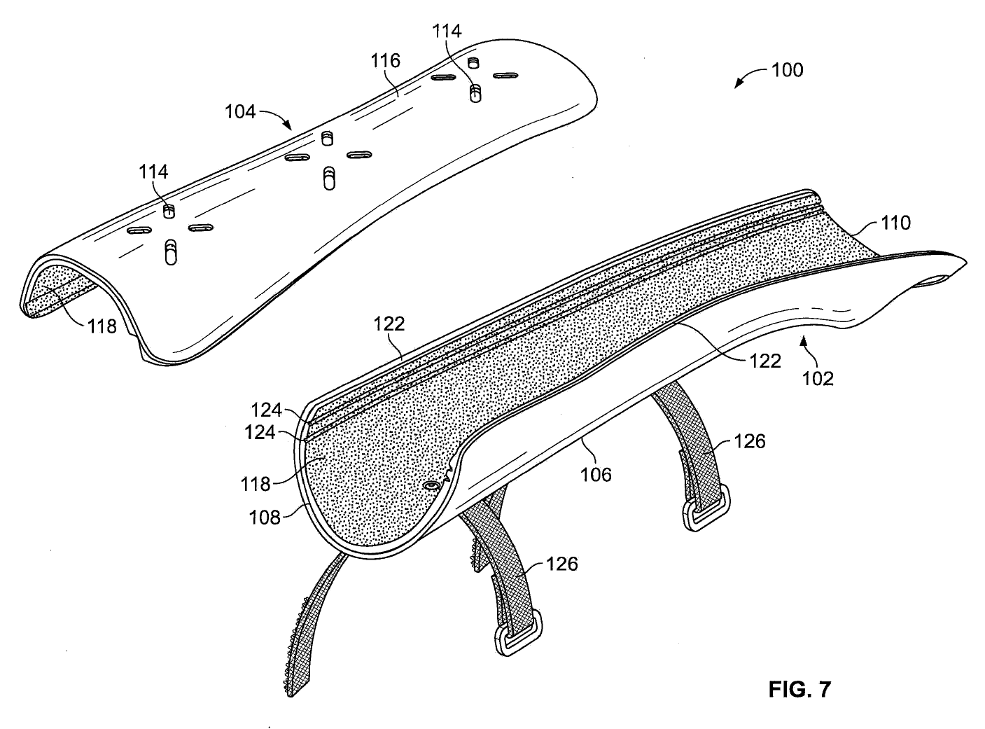

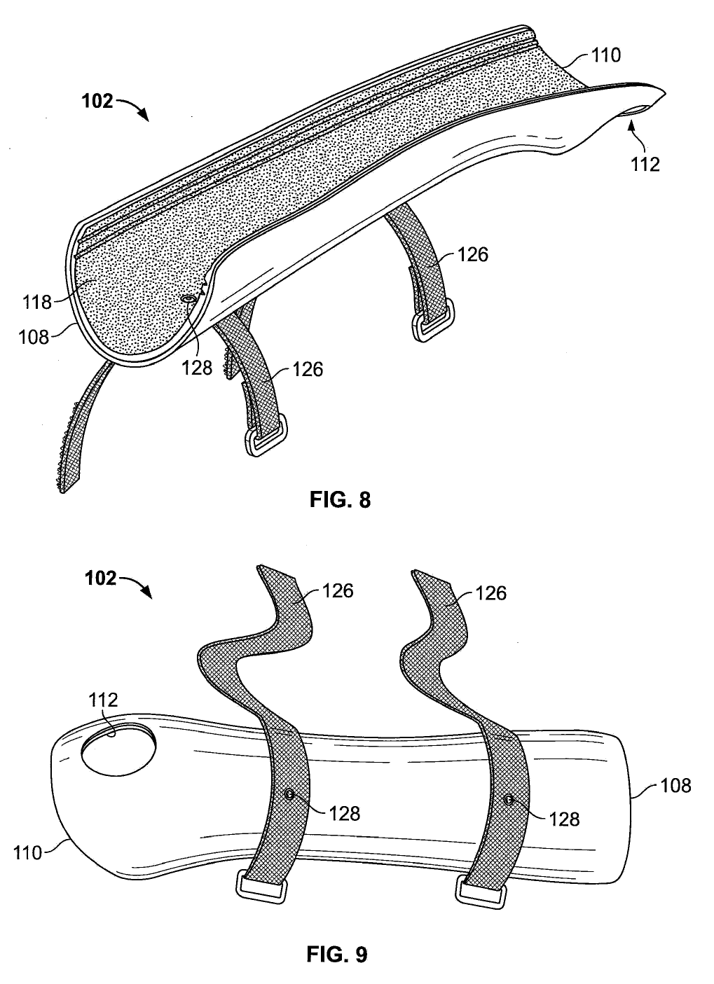

Turning to FIGS. 6 through 10, another brace according to the present teachings is illustrated applied to a patient appendage 2 and generally identified at reference character 100. As shown and similar to the brace 10, the brace 100 may be particularly adapted for use on a hand, wrist and forearm to promote healing. In other applications, the teachings may be adapted for other appendages of the body. As such, it will again be understood that the present teachings have application beyond the embodiment shown throughout the drawings.

The brace 100 may generally include a first member 102 and a second member 104. The first member may be a palmar member 102 and the second member may be a dorsal member 104. The first and second members 102 and 104 may be positioned on opposite sides of the appendage 2. As particularly shown in FIG. 6, the first member 102 may be positioned adjacent a palmar side of the hand and corresponding portions of the wrist and forearm. The first and second portions 102 and 104 may cooperate to circumferentially surround the forearm, wrist and hand.

The first member 102 may include an outer layer or shell 106. The outer shell 106 may be constructed of a substantially rigid plastic, such as polyethylene, or other material having acceptable strength and durability characteristics. The outer shell 106 may extend longitudinally between a first end or proximal end 108 and a second end or distal end 110. In a manner similar to the brace 10, the outer shell 106 is sized and shaped to receive the patient appendage 2 in a predetermined orientation. In this regard, the outer shell 106 may include a concavely curved inner surface for receiving the patient appendage 2. The second end 110 defines an opening 112 for receiving the patient thumb. As illustrated, the first member 102 may circumferentially extend approximately 180° about the appendage 2.

The second member 104 may be positioned adjacent a dorsal side of the hand and corresponding portions of the wrist and forearm. The second member 104 may include an outer shell 116 constructed of a substantially rigid plastic and may be formed to include one or more openings 114 to improve ventilation to the appendage 2. The openings 114 may be greater or smaller in number or size within the scope of the present teachings.

The inner surfaces of the outer shells 106 and 116 of the first and second members 102 and 104 may be provided with a breathable cushioning material 118. The cushioning material 118 may be adhesively or otherwise suitably secured to the outer shells 106 and 116 of the first and second members 102 and 104. While various materials may be employed, one suitable cushioning material is polyurethane foam having approximate durometer of two pounds.

The first and second members 102 and 104 may be provided with cooperating features to facilitate relative positioning. As perhaps most particularly shown in the cross-sectional view of FIG. 10, one of the first and second members 102 and 104 may define longitudinally extending ledges 120 for receiving and opposing longitudinally extending edges 122 of the other of the first and second members 102 and 104. In the embodiment illustrated, the second member 104 includes the longitudinally extending ledges 120 and the first member 102 nests within the second member 104.

One or both of the first and second members 102 and 104 may be trimmed to provide a proper anatomical fit with the appendage 2. The longitudinally extending edges 122 of the first member 102 may be provided with one or more trim lines 124 to guide trimming and maintain a proper mating engagement between the members 102 and 104. The trim lines 124 may be provided in the cushioning material 118 carried by the first member 102.

As illustrated, the brace 100 may include a strap arrangement for removably securing the members 102 and 104 to the appendage 2. The strap arrangement may include one or more straps 126. The straps 126 may be secured to the brace 100 with rivets 128 or in any manner well known in the art. The straps 126 may wrap circumferentially around the brace 100 and carry cooperating hook and loop or other contact closure material for fastening. Other manners for fastening may be alternatively employed. The straps 126 allow the brace 100 to be readily removed and re-secured to the appendage 2.

The brace 100 may additionally include a safety device 130 (see FIG. 6) for preventing inadvertent or unauthorized (e.g., by a child) removal of the brace 100. The safety device may include a safety strap 130 circumferentially surrounding the brace 100. The safety strap 130 may include a snap-type connector 132 that cannot easily be unsnapped. The safety strap 130 may be cut to be removed. Alternatively, the safety device may require a tool (e.g., key) for locking and unlocking.

With the relative positions of the first and second members 102 and 104 fixed, the brace may effectively behave as a single part. The brace 100 may have a certain stiffness generated through its geometry (i.e., its assembled geometric moment of inertia) that is greater than a stiffness generated by the geometry of traditional clamshell type braces.

The brace 100 may be provided in various sizes to accommodate various patient appendages 2. Different sizes may be employed depending on a particular anatomical measurement. In this regard, a wrist measurement taken directly proximal to the ulnar styloid may indicate an appropriate brace size.

In certain applications, it may be desirable to incorporate one or more fluid bladders (not shown) such as air bladders. The bladders can be selected from different sizes and inserted between the first and second members. The bladder(s) may be single or multi-compartmental members that can be inserted between the first and second members or affixed to one or both of the first and second members. The bladder(s) may be inflated to: a) provide an improved fit for the patient; b) fill in voids in between the brace and the appendage; and/or c) lock the first and second members together via a force fit exerted by the outward force of the bladder(s) on the first side on member which pushes the member against the inside surfaces of member thereby locking the two members together.

It will be understood that the present teachings provide brace assemblies 10 and 100 that may be pre-formed to accommodate an appendage 2 in a predetermined orientation. In this regard, the brace assemblies 10 and 100 can be configured to position the appendage 2 in a preferred orientation for proper healing. The brace assemblies 10 and 100 may also be easily removed and replaced to maintain proper hygiene. The brace assemblies 10 and 100 may also be constructed of waterproof materials that are not adversely affected by bathing and other everyday activities that may degrade conventional casting materials.

While specific examples have been described in the specification and illustrated in the drawings, it will be understood by those skilled in the art that various changes may be made and equivalence may be substituted for elements thereof without departing from the scope of the present teachings as defined in the claims. Furthermore, the mixing and matching of features, elements and/or functions between various examples may be expressly contemplated herein so that one skilled in the art would appreciate from the present teachings that features, elements and/or functions of one example may be incorporated into another example as appropriate, unless described otherwise above. Moreover, many modifications may be made to adapt a particular situation or material to the present teachings without departing from the essential scope thereof. Therefore, the scope of the present teachings includes any embodiments following within the foregoing description and any appended claims.

What is claimed is:

1. A brace for a patient appendage, the brace comprising:
a first member positionable proximate a first side of the patient appendage, the first member including a main body portion extending in a longitudinal direction from a proximal end to a distal end, and a pair of proximal circumferential extensions extending from the main body portion at the proximal end in a circumferential direction; and
a second member positionable proximate a second side of the patient appendage, the second member including a proximal end having an outer convexly curved portion, the convexly curved portion matingly received by inner sides of the proximal circumferential extensions of the first member.

2. The brace of claim 1, wherein the first member includes a pair of distal circumferential extensions extending circumferentially from the distal end of the main body.

3. The brace of claim 2, wherein the second member includes a distal end matingly received by the distal circumferential extensions.

4. The brace of claim 1, wherein the first and second members are cooperatively shaped to define openings in laterally opposing sides of the brace.

5. The brace of claim 1, wherein the pair of proximal circumferential extensions and the main body are unitary defining a complete circumference.

6. The brace of claim 1, wherein the pair of proximal circumferential extensions and the main body define a circumferential portion of more than 180 degrees.

7. The brace of claim 1, wherein each of the first and second members include a breathable cushioning inner layer.

8. The brace of claim 1, wherein the first member includes an aperture adapted to accommodate a separate portion of the patient's appendage.

9. A brace for an arm of a patient, the brace comprising:
a palmar member positionable proximate a palmar side of the patient arm, the palmar member including a main body portion extending in a longitudinal direction from a proximal end to a distal end, and a pair of proximal circumferential extensions extending from the main body portion at the proximal end in a circumferential direction; and
a dorsal member positionable proximate a dorsal side of the patient arm, the dorsal member including a proximal end having an outer convexly curved portion, the convexly curved portion matingly received by inner sides of the proximal circumferential extensions of the palmar member.

10. The brace of claim 9, wherein the palmar member includes a pair of distal circumferential extensions extending circumferentially from the distal end of the main body.

11. The brace for a patient arm of claim 10, wherein the dorsal member includes a distal end matingly received by the circumferential extensions of the palmar member.

12. The brace for a patient arm of claim 7, wherein the palmar member defines a recess for receiving a thumb of the patient.

13. The brace for a patient arm of claim 12, therein the recess is a hole passing through the palmar member.

14. The brace for a patient arm of claim 9, wherein the palmar and dorsal members are cooperatively shaped to define openings in laterally opposing sides of the brace.

15. The brace for a patient arm of claim 9, wherein the pair of proximal circumferential extensions and the main body are unitary defining a complete circumference.

16. The brace of claim 9, wherein the pair of proximal circumferential extensions and the main body define a circumferential portion of more than 180 degrees.

17. The brace of claim 9, further comprising a lockable element for securing the palmar and dorsal members relative to one another.

18. A brace for an arm of a patient, the brace comprising:
a palmar member positionable proximate a palmar side of the patient arm, the palmar member including a thumb aperture, an outer waterproof layer, and an inner layer of breathable cushioning material;
a dorsal member positionable proximate a dorsal side of the patient arm, the dorsal member including a plurality of ventilating apertures, an outer waterproof layer and an inner layer of breathable cushioning material; and
at least one longitudinal marking trimmable to size, the marking defined in a longitudinally extending ledge of one of the palmar and dorsal members.

19. The brace of claim 18, wherein the brace further comprises:
at least one closure member attached to one of the palmar and dorsal members adapted for securing the brace; and
at least safety device adapted for preventing removal of the brace.

20. The brace of claim 18, wherein the markings are defined in the cushioning material of the first member.
